(19) United States
(12) Patent Application Publication
(10) Pub. No.: US 2023/0130742 A1
(43) Pub. Date: Apr. 27, 2023

(54) METHOD FOR LONG-DISTANCE TRANSMISSION OF PHYSIOLOGICAL SIGNALS IN A CLOSED LOOP SYSTEM

(71) Applicant: ExeBrain Co., Ltd., Taipei City (TW)

(72) Inventors: Shu-Chun Kuan, Taipei City (TW); Jason Chun-Cheng Lin, Taipei City (TW); Chin-Yeh Lu, Taipei City (TW)

(21) Appl. No.: 17/872,041

(22) Filed: Jul. 25, 2022

(30) Foreign Application Priority Data
Oct. 21, 2021 (TW) 110139162

(51) Int. Cl. A61B 5/00; A61B 5/369

(52) U.S. Cl. CPC A61B 5/0006; A61B 5/369; A61B 5/6803

(57) ABSTRACT

Provided is a method for long-distance transmission of physiological signals in a closed loop system, including generating a signal at a user terminal of the closed loop system, compressing the signal to generate a compressed signal, transmitting the compressed signal from the user terminal to a computing terminal of the closed loop system, receiving and comparing the compressed signal with a database at the computing terminal to generate a comparison result and a feedback signal, and transmitting the feedback signal from the computing terminal to the user terminal. A time interval between generating the signal and receiving the feedback signal at the user terminal is less than a threshold.

### CROSS-REFERENCE TO RELATED APPLICATION

This application claims the priority of Taiwanese patent application No. 110139162, filed on Oct. 21, 2021, which is incorporated herewith by reference.

### BACKGROUND OF THE INVENTION

### 1. Field of the Invention

The present invention relates to a method for long-distance transmission of physiological signals, especially a long-distance bidirectional communication processing system and method for physiological signals in a closed loop system.

### 2. The Prior Arts

The existing biological feedback training mainly uses a wireless device at the input terminal, such as a pair of electrode patches, to compare brain wave changes before and after for the three areas of the parietal lobe, and to detect neurophysiological feedback by the influence of sensorimotor rhythm (SMR), or to collect physiological signals and upload physiological data to the cloud platform via wired or wireless transmission modules for analysis. The user needs to open the APP or related applications to read the physiological device during sleep in retrospect manner. However, the prior art usually prevents the user from obtaining physiological information such as brain waves or heartbeat variability immediately, and the user needs to wait for several hours to several days for analysis.

Therefore, there is a need to propose improved methods and systems that can provide real-time feedback remotely, allow the user to immediately receive the result, and the user is allowed to adjust physiological signals for recovery through visual or auditory feedback.

### SUMMARY OF THE INVENTION

In order to solve the aforementioned problem, the present invention provides a long-distance bidirectional transmission processing system for physiological signals, which includes a user terminal including: a brain wave cap, a processing unit, an output unit, and a remote transmission module, wherein the processing unit receives a brain wave physiological signal of a user's brain detected by the brain wave cap, the processing unit selects one of a plurality of signal transmission modes to process the brain wave physiological signal and transmits the brain wave physiological signal to a cloud network through the remote transmission module, and the processing unit receives a comparison feedback signal through the remote transmission module, and the output unit outputs a comparison result of the comparison feedback signal; and a computing terminal including: a cloud server and a brain wave physiological database, wherein the cloud server receives the brain wave physiological signal from the cloud network, and selects one of the plural signal comparison modes to process the brain wave physiological signal, generates the comparison feedback signal according to the brainwave physiological database, and transmits the comparison feedback signal to the user terminal. When the user terminal selects one of the plurality of signal transmission modes to process and transmit the brain wave physiological signal, the computing terminal selects one of the plurality of signal comparison modes to process the brain wave physiological signal and transmits the comparison feedback signal simultaneously.

According to an embodiment of the present invention, the user uses a prediction compression to generate a combination result through a corresponding relationship between the waveforms of the physiological signal to compress the physiological signal.

According to an embodiment of the present invention, the user terminal uses a numerical compression to compress the physiological signal according to a difference vector angle and a value of difference vector between consecutive points on a waveform of the physiological signal in the same channel.

According to an embodiment of the present invention, the user terminal uses a block compression to compress the physiological signal by assuming the physiological signal with the same characteristics as a coded data.

According to an embodiment of the present invention, the user terminal uses a shape compression, which compresses the physiological signal through a static base value of picture and a picture displacement of difference between the waveforms of the physiological signal in different channels.

In order to solve the aforementioned problem, the present invention also provides a closed loop system, which includes a user terminal and a computing terminal. The user terminal generates a signal and compresses the signal to generate and transmit a compressed signal. The computing terminal receives and compares the compressed signal to generate and send a feedback signal to the user terminal. A time interval between the user generating the signal and receiving the feedback signal is less than a threshold.

By using the system of the present invention for processing the remote bidirectional communication of the physiological signal, the evaluation efficiency can be improved, and the biological feedback training system can achieve remote real-time feedback, so that the user immediately receive the result, and the user can immediately understand the condition, and the user can adjust the physiological signal for recovery through the feedback.

### BRIEF DESCRIPTION OF THE DRAWINGS

FIG. 1 is a schematic diagram of a closed loop system according to an embodiment of the present invention; and

FIGS. 2 to 5 are schematic diagrams of compression methods according to different embodiments of the present invention.

### DETAILED DESCRIPTION OF THE PREFERRED EMBODIMENT

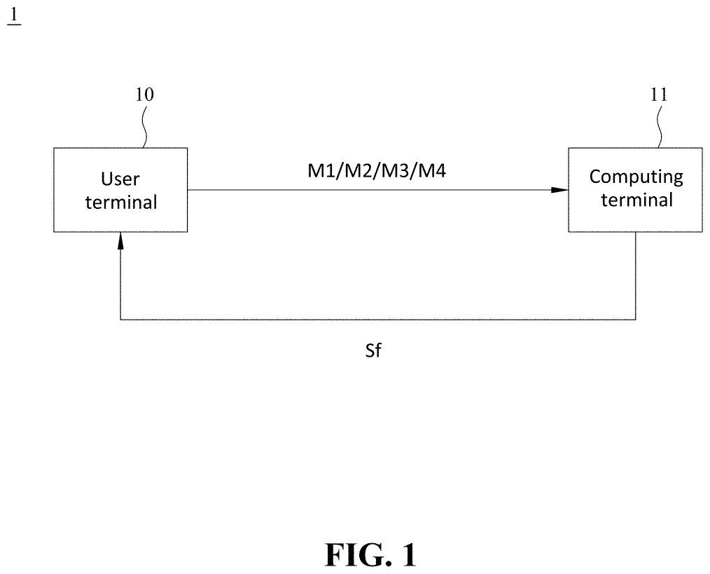

Please refer to FIG. 1, FIG. 1 is a schematic diagram of a closed loop system 1 according to an embodiment of the present invention. As shown in FIG. 1, the system 1 includes a user terminal 10 and a computing terminal 11. The user terminal 10 compresses and transmits the compressed signal to the computing terminal 11 through the compression mode M1/M2/M3/M4, and then the computing terminal 11 returns a feedback signal Sf to the user terminal 10. The used method for long-distance transmission of the physiological signals includes the following steps: a signal is generated at the user terminal 10 of the closed loop system 1, and the signal is compressed to generate a compression signal; the compressed signal is transmitted from the user terminal 10 to the computing terminal 11 of the system 1; the compressed signal is received at the computing terminal 11 and compared with a database to generate a comparison result and the feedback signal Sf through machine learning and artificial intelligence, so as to reduce the data transmission between the computing terminal 11 and the user terminal 10. For example, when the system 1 is used for biological feedback training, a biological indicator of the signal will be compared with a detection database including brain wave and heart rate variability data to generate a comparison result and the feedback signal Sf; and the computing terminal 11 transmits the feedback signal Sf to the user terminal 10. The time interval between the user terminal 10 generating the signal and receiving the feedback signal Sf is less than a threshold value, the threshold value is usually less than 3 seconds, but can also be set as 5 seconds, 10 seconds, 20 seconds, 30 seconds, and no more than 30 seconds.

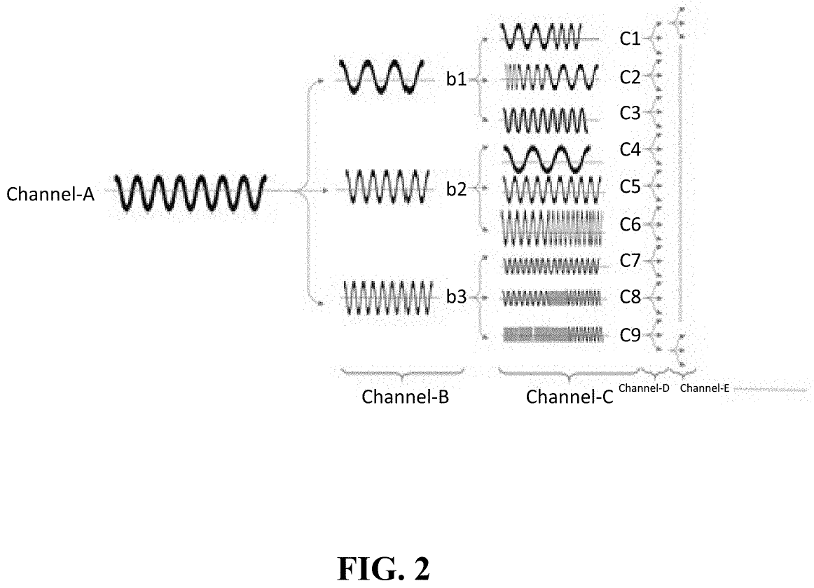

FIGS. 2 to 5 are schematic diagrams of compression methods according to different embodiments of the present invention corresponding to the compression mode M1/M2/M3/M4. As shown in FIG. 2, in this embodiment, the user terminal 10 uses the compression mode M1, which uses a prediction (probability) calculation compression to compress the signal by generating a combination result through a corresponding relationship between the waveforms of the signal. Corresponding similar waveforms b1, b2, b3, etc. will appear in channels Channel-A and Channel-B. Corresponding similar waveforms c1, c2, c3 . . . etc. will appear in channels Channel-B and Channel-C. The compression method in FIG. 2 mainly compare the feedback of the user terminal 10 and the computing terminal 11, so as to reduce the data transmission between the computing terminal 11 and the user terminal 10. The feedback signal Sf is compared through a machine learning and artificial intelligence method to find the most suitable mode and generate feedback. Due to the physical limitations of the human body (nerve conduction, muscle operation, etc. are all within a certain range and affect each other), the difference between the channels and the compression method needs to be analyzed for a long time through the database. The above-mentioned waveform is a waveform with a greater probability of appearance. If a waveform different from the above-mentioned waveform appears, the different waveform will be defined as noise or be ignored. Therefore, when the signal transmitted in this mode is transmitted and compared, the waveform with the greatest probability to appear will be predicted and further calculated to generate the feedback signal Sf. For example of a pinball table, after a marble is sent out, it will go left or right when it encounters the first nail, but the force of the initial ejection can be used to predict the encountering of the first nail and then the second nail. Finally, this marble will definitely fall in the groove at the bottom, the probability of exceeding the groove is very small and affected by other irresistible factors. After receiving the brain wave signal from the user terminal, there are different positions on the brain wave cap. As to a 19-channel brain wave cap used, the brain wave of the channel Channel-A may have different probability b1, b2, b3 . . . bx distributed after spectrum analysis. If the brain wave signal with probability b2 from the channel Channel-A is received on the channel Channel-B, then the brain wave signal with probability c5 from the channel Channel-B may be received on the channel Channel-C. Similarly, such a mode will fall into the database of a type of certain behavioral performance or mental characteristics.

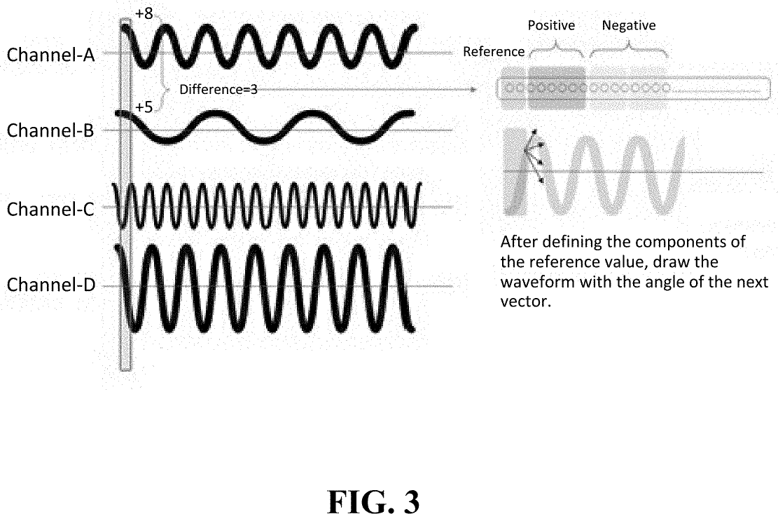

As shown in FIG. 3, in this embodiment, the user terminal 10 uses a compression mode M2, which uses a numerical compression, and a difference vector angle and a value of difference vector between consecutive points on a waveform of the signal in the same channel are used to compress the signal. The object of difference comparison refers to the comparison between the sampled values at consecutive time points. The sampled value at the time point t(n+k) is compared with the sampled value at the time point t(n). When the value of k is greater, the difference between the two sampling time is greater. When the waveform changes little, the increase of the k value will make the compression ratio increase and the transmission value decrease. The positioning of the reference value here is used to predict the angle and value of the vector to draw the waveform. The channel Channel-A defines vector 1, the channel Channel-B defines vector 2, and the channel Channel-C defines vector 3 . . . and so on. For the example of binary, as 2³=8, there are eight variations “000”, “001”, “010”, “100”, “011”, “101”, “110”, and “111”. When the physiological signals of the brain wave are captured, a specific number of bits is given as a reference value, and the subsequent brain waves can be described by a difference vector. Therefore, when the physiological signal or brain wave signal of the user is collected, after the feature of the factor is detected, the variation of the vector angle of the subsequent data and the variation of the positive and negative values are used to compare the characteristics of the subsequent physiological signals. The compression mode M2 in FIG. 3 mainly uses the difference of each sampling in individual channels, and the compression mode M2 can be used in each channel, and the compression mode M1 in FIG. 2 uses the comparison between different channels.

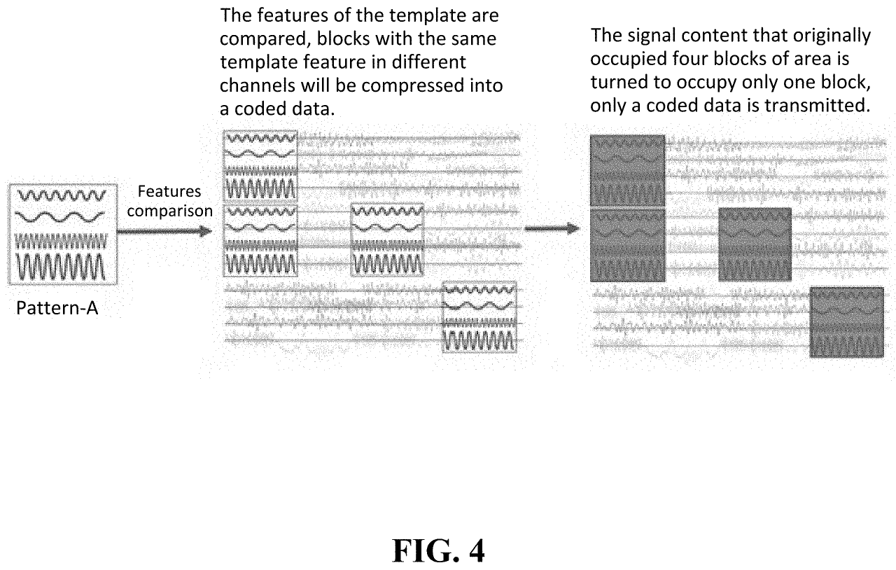

As shown in FIG. 4, in this embodiment, the user terminal 10 uses a compression mode M3, which uses a block compression, and compresses the signal with the same characteristic as the same coded data. The pattern of feature here is defined for the signal, a large number of signals are divided into blocks, and a template feature is defined for the block, such as a template feature Pattern-A. Blocks with the same template feature in different channels will be compressed into a coded data, so that the same template feature will be transmitted with the same coded data during the transmission and calculation process. In FIG. 4, the blocks that meet the template feature Pattern-A will be represented by the same coded data. The signal content that originally occupied four blocks of area is turned into only one image with a 4:1 compression ratio.

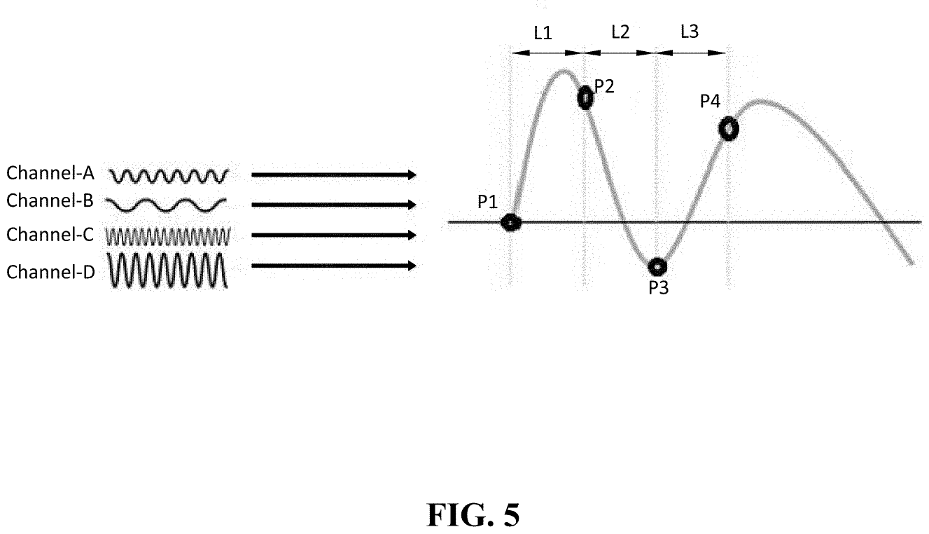

As shown in FIG. 5, in this embodiment, the user terminal 10 uses the compression mode M4, which uses a compression using three segments divided by four points, and uses specific frequencies sampled for the signal waveforms on different channels Channel-A to Channel-D. In this embodiment, a mark is made for every four points, and three segments are generated for every four points. The features on the three segments can be extracted, the four points P1-P4 and the features of the three segments corresponding to the horizontal sections L1-L3 can be used to rebuild the original waveform. In this way, the computing terminal 11 can be compared with the database by transmitting the four points and the features of the three segments.

In the present invention, after the user terminal 10 transmits a signal, the signal needs to be compared with a database. The comparison can be performed on the computing terminal 11, and then the feedback signal Sf needs to be sent back to the user terminal 10, and the user terminal 10 also continues to generate brain waves or other physiological signals at the same time, and form a closed-loop feedback mechanism. The user terminal 10 actually compares the signals at the same time as the signal is generated and receives the feedback signal Sf at the same time to be adjusted. For example, when the subject's brain waves are compared with the database, if the ratio of brain waves having a pattern with certain feature reaches a threshold, the subject will be given sound or visual feedback through the mobile phone, TV or computer screen, and allowed to understand the current state of brain wave, the real-time state of the brain waves of the subject is converted into sound or visual feedback signals, so as to achieve the purpose of brain training. Therefore, in comparison technology used during the transmission and calculation, the efficiency of transmission is higher and the efficiency of comparison is faster. The above-mentioned M1/M2/M3/M4 mode used in the present invention for signal transmission and comparison can quickly provide the user with the feedback of physiological signals, especially the characteristics of brain waves during comparison and calculation, and the permutations of the brain regions and patterns included in the signals can be represented by as many as millions to billions of possible waveforms. For example, the present invention can be used to select the most suitable comparison strategy for the M1/M2/M3/M4 model according to different ethnic groups (demographic variables such as gender, age, education level, etc.), different behaviors or mental functions. As to s subject with memory degradation problems, the best comparison strategy may subsequently use modes M4, M1, M2, and M3 in order. As to a subject with symptoms of attention deficit hyperactivity, the best comparison strategy may subsequently use modes M2, M1, M4, and M3 in order. As to a subject in need of improving the exercise reflection, the best comparison strategy may use mode M1.

The method for long-distance transmission of physiological signals in a closed loop system of the present invention makes the long-distance transmitted signals undistorted and transmitted back after compared. General physiological signal (such as EEG) is more complex and usually takes some time to calculate and generate the result, while the method of the present invention can transmit, compare and feedback faster, and the time spent must be within allowable range (the present invention is object to minimize the delay to be less than 3 seconds, and 30 seconds is a permissible delay), so the efficiency of evaluation can be improved. The biological feedback training system achieves remote real-time feedback, so that the subject can immediately understand the conditions, and can adjust the physiological signals for recovery through feedback.

The present invention is not limited to the above-mentioned embodiments. It is obvious to those skilled in the art that various modifications and variations can b e made to the present invention without departing from the spirit or scope of the present invention.

Therefore, the present invention is intended to cover the modifications and variations made to the present invention or falling within the scope of claims and the equivalent scope.

What is claimed is:

1. A long-distance bidirectional transmission processing system for physiological signals in a closed loop system, comprising:
a user terminal including: a brain wave cap, a processing unit, an output unit, and a remote transmission module, wherein the processing unit receives a brain wave physiological signal of a user's brain detected by the brain wave cap, the processing unit selects one of a plurality of signal transmission modes to process the brain wave physiological signal and transmits the brain wave physiological signal to a cloud network through the remote transmission module, and the processing unit receives a comparison feedback signal through the remote transmission module, and the output unit outputs a comparison result of the comparison feedback signal; and
a computing terminal including: a cloud server and a brain wave physiological database, wherein the cloud server receives the brain wave physiological signal from the cloud network, and selects one of the plural signal comparison modes to process the brain wave physiological signal, generates the comparison feedback signal according to the brainwave physiological database, and transmits the comparison feedback signal to the user terminal;
wherein when the user terminal selects one of the plurality of signal transmission modes to process and transmit the brain wave physiological signal, the computing terminal selects one of the plurality of signal comparison modes to process the brain wave physiological signal and transmits the comparison feedback signal simultaneously.

2. The long-distance bidirectional transmission processing system according to claim 1, wherein the user uses a prediction compression to generate a combination result through a corresponding relationship between the waveforms of the physiological signal to compress the physiological signal.

3. The long-distance bidirectional transmission processing system according to claim 1, wherein the user terminal uses a numerical compression to compress the physiological signal according to a difference vector angle and a value of difference vector between consecutive points on a waveform of the physiological signal in the same channel.

4. The long-distance bidirectional transmission processing system according to claim 1, wherein the user terminal uses a block compression to compress the physiological signal by assuming the physiological signal with the same characteristics as a coded data.

5. The long-distance bidirectional transmission processing system according to claim 1, wherein a mark is used to represent four given points, and three intervals are generated for the four given points, three line segment features can be extracted from the three intervals.
